(19) United States
(12) Patent Application Publication
(10) Pub. No.: US 2025/0360546 A1
(43) Pub. Date: Nov. 27, 2025

(54) A SYSTEM AND METHOD FOR ANAEROBIC DRY DIGESTION OF LIGNOCELLULOSIC BIOMASS

(71) Applicants: QUBE RENEWABLES LTD, Somerset (GB); BLUE PLANET ENVIRONMENTAL SOLUTION PTE LTD, Singapore (SG)

(72) Inventors: Mark CLAYTON, Somerset (GB); Joanna CLAYTON, Somerset (GB)

(21) Appl. No.: 19/114,685

(22) PCT Filed: Sep. 30, 2023
(86) PCT No.: PCT/IB2023/059824

§ 371 Date: Mar. 24, 2025

(30) Foreign Application Priority Data
Sep. 30, 2022 (IN) 202211019039

(51) Int. Cl. B09B 3/65; C12M 1/00; C12M 1/107

(52) U.S. Cl. CPC B09B 3/65; C12M 21/04; C12M 23/36; C12M 29/00; C12M 47/18

(57) ABSTRACT

The present disclosure is directed to a system for anaerobic dry digestion of lignocellulosic biomass including: a dry digester having a ground sheet and a cover for the lignocellulosic biomass. The digester generates leachate and biogas from anaerobic digestion. The system includes an irrigation/port system for wetting of the lignocellulosic biomass under the cover; a sump system for monitoring the leachate; a rotary valve-connected at least to the pump and the dry digester; a gas storage bladder for collecting the biogas, the gas storage bladder connected at least to a gas treatment unit and a gas booster pump; and a hi-rate digester tank connected at least to the rotary valve for receiving the leachate therefrom.

### FIELD OF THE INVENTION

The present disclosure relates to a system and method for anaerobic dry digestion of lignocellulosic biomass.

### BACKGROUND

Harvesting and processing agricultural crops produce by-products which are considered as agricultural residue or agricultural waste. These are non-wood lignocellulosic materials and are rich source of cellulose with lignin. These may include stalk, cane, seed pod, and leaves.

Worldwide, it is a common practice to dump these agricultural residues as waste or garbage. The piles of residue decompose by microbial activity and become a nuisance for the environmental health. Further, traditionally, agricultural waste has been disposed of either by composting or by burning in the fields. They have been burnt for cheap energy generation and have been a source of air pollution, due to which their use for energy generation is not advisable. Accordingly, proper utilization of these agricultural wastes is required as they are rich in cellulose.

Rice, wheat, sugarcane, soybean, corn, banana, pineapple, bamboo, and okra are few examples of crops that generate considerable agricultural residues. These contribute to a majority of the total annual production of biomass residue and are an important source of cellulosic content as well.

To promote sustainable living, reversing the adverse effects of climate change, affordable and clean energy alternatives, and efficient waste management are highly desired. In this regard, fibrous agricultural waste based on lignocellulose can be converted into biogas and fertilizer by anaerobic fermentation utilizing dry digestion techniques.

The standard dry digestion techniques, conventionally known, suffer from several drawbacks. For instance, the requirement of large concrete bunkers results in very high capital expenditure (capex). Further, these bunkers lack mobility owing to the use of concrete and hence, cannot be moved from one location to another, thereby limiting their utility to a specific location. Moreover, current dry digesters demand a substantial parasitic power load for agitating and moving feedstocks within the system. The vital biological processes essential for dry digestion are disrupted during each batch change, resulting in prolonged batch retention times, and reduced overall efficiency. Furthermore, there is a constant requirement for specialist technology for loading/unloading these digesters.

Thus, there is a need for a more viable solution for anaerobic dry digestion of lignocellulosic biomass which is compact and can be easily dismantled and reassembled onsite (has mobility), reduces the high capex requirement, and environmental impact, overcomes the inconsistent biology (and therefore enables more efficient biogas production) through different batches, reduction in high parasitic power use, and long retention times.

### SUMMARY

In one aspect, the present disclosure is directed to a system for anaerobic dry digestion of lignocellulosic biomass. The system includes a dry digester comprising a ground sheet and a cover for the lignocellulosic biomass. The dry digester generates leachate and biogas from anaerobic digestion. The system also includes an irrigation/port system for wetting of the lignocellulosic biomass under the cover. The irrigation/port system comprises a plurality of irrigation lines disposed over the cover and configured to: introduce a liquid suspension into the dry digester thereby wetting the lignocellulosic biomass, the liquid suspension comprising of a microbial community, nutrients, buffering agents, and water; and recycle the leachate. The system further includes a sump system for monitoring the leachate, and which comprises: an external tank connected to the dry digester for storing the leachate; a sump tank connected to the external tank for storing the leachate; and a pump connected to the external tank and the sump tank. The pump circulates the leachate from the sump tank. Further, a rotary valve connected at least to the pump and the dry digester is also provided in the system. The rotary valve recycles the leachate from the sump tank to the dry digester. A gas storage bladder for collecting biogas is also provided. The gas storage bladder connects at least to a gas treatment unit and a gas booster pump. The gas booster pump connects at least to the dry digester and pumps biogas through the gas treatment unit. The system further includes a hi-rate digester tank connected at least to the rotary valve for receiving the leachate therefrom. The hi-rate digester tank comprises a bio-media for improving biogas offtake from the leachate.

In an example embodiment, the dry digester comprises: a plurality of tubes disposed on the ground sheet for collecting water and/or leachate. The plurality of tubes is connected with a sump return box for storing the collected water and/or leachate. The dry digester also includes a plurality of ground sheet poles and a plurality of cover sheet poles installed on the ground sheet. Each of the ground sheet pole and each of the cover sheet pole provide structural stability to the dry digester. The dry digester further includes webbing over the cover for providing tautness to the dry digester. The webbing is formed by a plurality of ropes and binds at least the plurality of ground sheet poles with the plurality of cover sheet poles across the ground sheet, thereby preventing escape of biogas and leakage of the leachate.

In another example embodiment, a ballast tube B is disposed on the cover and the ground sheet. The ballast tube B is dispersed on a periphery of the dry digester on the ground sheet and the cover, thereby acting as a weight to the cover and the ground sheet and sealing the dry digester to prevent leakage of biogas.

In another example embodiment, the dry digester is maintained at a temperature ranging between 20° C. to 40° C., and pH ranging between 6.0 to 8.0.

In still another example embodiment, the lignocellulosic biomass is stacked on the ground sheet to form a pile of lignocellulosic biomass having a top surface and a bottom surface. The cover encloses the pile from the top surface to the bottom surface on the ground sheet.

In yet another example embodiment, the lignocellulosic biomass is devoid of size reduction before stacking in the dry digester.

In still another example embodiment, the microbial community comprises of hydrolytic bacteria, fermentative bacteria, obligate hydrogen producing bacteria, homoacetogenic bacteria, syntrophic acetate oxidising bacteria, acetoclastic methanogens, hydrogenotrophic methanogens, and denitrifying methanogens.

In a further example embodiment, the wetting allows the microbial community to uniformly distribute within the lignocellulosic biomass, thereby ensuring complete breakdown of the lignocellulosic biomass into biogas via hydrolysis, acidogenesis, acetogenesis, and methanogenesis.

In a still further example embodiment, lignocellulosic biomass is devoid of prior heating and/or prior mixing before stacking in the dry digester.

In yet another example embodiment, the biomedia comprises extruded bioplastic.

In still another example embodiment, the leachate from the dry digester is transported to the external tank and/or the sump tank under the influence of gravity.

In a further example embodiment, the Hi-rate digester tank is heated at a temperature ranging between 15° C. to 50° C., thereby resulting in an overflow of the leachate, said leachate being transported back to the sump tank.

In a still further example embodiment, the system further comprises a condensate remover connected to the dry digester and the gas booster pump. The condensate remover receives biogas at least from the dry digester and the Hi-rate digester tank for removing moisture present in the biogas.

In another example embodiment, the ground sheet and the cover, independent of each other, comprise a fabric material.

In yet another example embodiment, the fabric material is selected from the group consisting of butyl, polyethylene, and polyvinyl chloride.

In another aspect, the present disclosure is directed to a method for anaerobic dry digestion of lignocellulosic biomass. The method includes the steps of: wetting, in a dry digester, the lignocellulosic biomass in the presence of a liquid suspension introduced by a plurality of irrigation lines disposed over a cover of the dry digester, thereby generating a leachate and biogas, wherein the dry digester is maintained at a temperature ranging between 20° C. to 40° C., and pH ranging between 6.0 to 8.0; monitoring, in a sump system comprising an external tank and a sump tank (124), the leachate stored in the external tank and the sump tank, the leachate being transported to the external tank and the sump tank under the influence of gravity; recycling, by a rotary valve, the leachate from the sump tank to the dry digester, the leachate being introduced in the dry digester by the plurality of irrigation lines. The method also includes the steps of: treating, in a hi-rate digester tank, the leachate received from the rotary valve in the presence of a bio-media and at a temperature ranging between 15° C. to 50° C., thereby improving the biogas offtake from the leachate; collecting, in a gas storage bladder, biogas received from the dry digester; and pumping, by a gas booster pump, biogas to a gas treatment unit, thereby removing at least hydrogen sulphide from the biogas.

In an example embodiment, in the dry digester optionally seeding is carried out after the wetting of the lignocellulosic biomass, the seeding being carried out in the presence of a seed material comprising manure.

In another example embodiment, the liquid suspension comprises of a microbial community, nutrients, buffering agents, and water.

In yet another example embodiment, the leachate is monitored for one or more of parameters selected from pH, temperature, dissolved solids, micro elements, and macro elements.

In a further example embodiment, the lignocellulosic biomass is devoid of prior heating and/or prior mixing before wetting.

In a still further example embodiment, prior to collecting biogas in the gas storage bladder, biogas received at least from the dry digester and the Hi-rate digester tank is subjected to moisture removal, in a condensate remover.

In further example embodiment, lignocellulosic biomass is selected from rice straw, wheat straw, bagasse, corn stover, Miscanthus, Napier grass, and palm fronds.

In another example embodiment, biogas comprises methane and carbon dioxide in a volume ratio ranging between 50:50 to 60:40.

### BRIEF DESCRIPTION OF THE DRAWINGS

Reference will be made to embodiments of the invention, examples of which may be illustrated in accompanying figures. These figures are intended to be illustrative, not limiting. Although the invention is generally described in context of these embodiments, it should be understood that it is not intended to limit the scope of the invention to these particular embodiments.

FIG. 1 illustrates a system for anaerobic dry digestion of lignocellulosic biomass in accordance with an embodiment of the present invention.

FIG. 2 shows a dry digester in accordance with an embodiment of the present invention.

FIG. 3 shows a cross-sectional view of the dry digester in accordance with an embodiment of the present invention.

FIG. 4 illustrates a cross-sectional view of the dry digester of FIG. 3 including lignocellulosic biomass and liquid level in accordance with an embodiment of the present invention.

FIG. 5 illustrates a method for anaerobic dry digestion of lignocellulosic biomass in accordance with an embodiment of the present invention.

FIG. 6 shows biogas yield in accordance with an embodiment of the present invention.

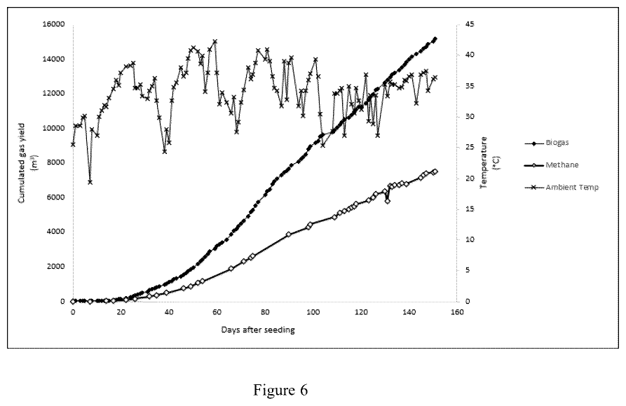

### DETAILED DESCRIPTION

Various features and embodiments of the present invention here will be discernible from the following further description thereof, set out hereunder.

The present disclosure is directed towards a system and a method for anaerobic dry digestion of lignocellulosic biomass for converting the lignocellulosic biomass into biogas and fertilizer. The biogas is further converted into usable fuels.

In one aspect, the present disclosure relates to a system for anaerobic dry digestion of lignocellulosic biomass.

In the present context, “anaerobic digestion” refers to a biologically mediated and controlled process for the degradation and conversion of lignocellulosic organic materials into biogas, digestate, and other valuable byproducts in an oxygen-deprived or anaerobic environment. This process is characterized by the absence of molecular oxygen (O₂) as a terminal electron acceptor during microbial metabolism. The “anaerobic digestion of lignocellulosic biomass” as described herein encompasses innovative methods, systems, and configurations tailored to the efficient and environmentally sound conversion of lignocellulosic feedstocks into biogas and other valuable commodities, while mitigating environmental impacts.

Herein, the term “leachate” refers to a liquid effluent or discharge generated during the anaerobic digestion process. The leachate is characterized by its composition, which comprises water-soluble compounds, byproducts, and residual organic and inorganic constituents derived from the lignocellulosic biomass feedstock. Furthermore, leachate may also comprise water along with the aforementioned ingredients.

Further, the term “digestate” refers to the residual material resulting from the anaerobic digestion process. The term encompasses both solid and liquid fractions of the post-digestion residue, which retain distinct organic, inorganic, and microbial constituents derived from the original lignocellulosic biomass feedstock. Typically, the digestate from anaerobic digestion has a considerably high total solid content and therefore, finds various applications, such as but not limited to fertilizer, animal bedding, compost production, and the likes. The specific use of digestate depends on its composition, the requirements of the local agricultural or waste management systems, and any regulatory guidelines governing its handling and application.

Furthermore, the phrase “lignocellulosic biomass” refers to a diverse class of renewable organic materials primarily composed of plant-derived components, namely cellulose, hemicellulose, and lignin. Lignocellulosic biomass constitutes a wide array of feedstock sources characterized by their structural complexity and their potential suitability for anaerobic digestion processes. The term “lignocellulosic biomass,” as expounded herein, encompasses a broad category of plant-derived organic materials characterized by their cellulose, hemicellulose, and lignin constituents. Innovations related to the anaerobic digestion of lignocellulosic biomass, its pretreatment, and its sustainable utilization are encompassed within the scope of the present invention.

Suitable examples of lignocellulosic biomass include rice straw, wheat straw, bagasse, corn stover, Miscanthus, Napier grass, and palm fronds. However, other similar lignocellulosic biomass, known to a person skilled in the art, may also be used as suitable feedstock for anaerobic digestion in the present invention.

Additionally, the phrase “dry digestion” in the present context refers to anaerobic digestion process specifically designed for the conversion of lignocellulosic biomass into biogas, leachate, and digestate in an environment characterized by low moisture content, typically below 35% by weight. Dry digestion is distinguished from wet or liquid-phase anaerobic digestion processes, which operate with significantly higher moisture levels.

Furthermore, in the present context “lignocellulosic biomass” may be interchangeably referred to as “biomass” or “substrate”.

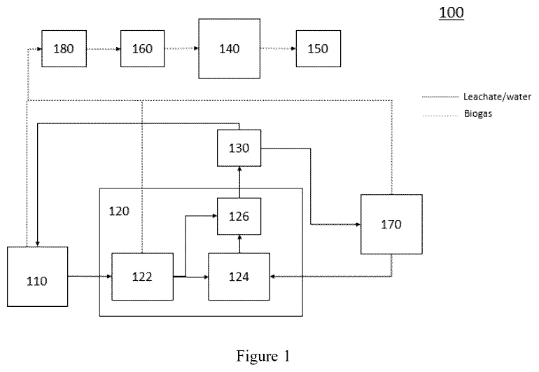

As shown in FIG. 1, the components of system 100 include a dry digester 110, an irrigation/port system 113 (not shown in Figures), a sump system 120, a rotary valve 130, a gas storage bladder 140, and a hi-rate digester tank 170. Each of these components are described in detail hereinbelow.

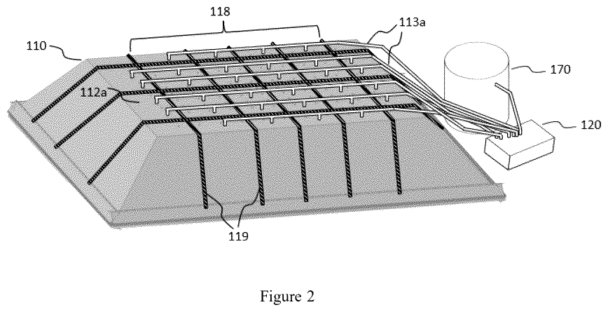

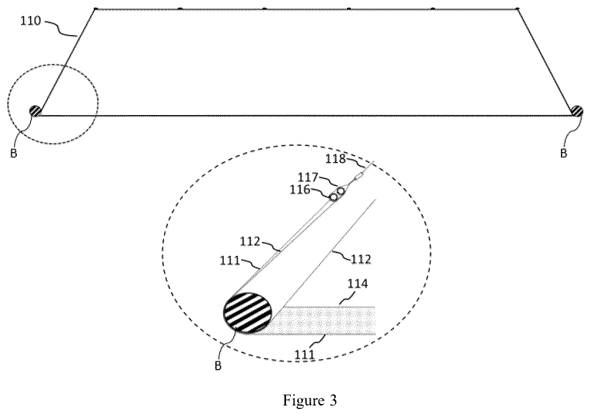

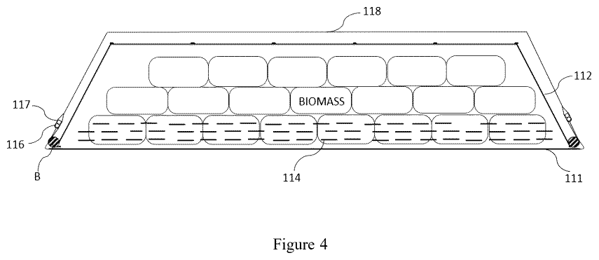

The dry digester 110 comprises a ground sheet 111 and a cover 112. The cover 112 forms an outermost layer 112a of the dry digester 110, and is disposed under the ground sheet 111, as shown in FIGS. 3 and 4. The biomass is stacked on the ground sheet 111 to form a pile or stack of biomass having a top surface and a bottom surface. The cover 112 encloses the pile from the top surface to the bottom surface on the ground sheet 111, thereby forming a majority portion of the dry digester 110, as shown in FIG. 2.

In an embodiment, the cover 112 fully encloses a topmost layer of the biomass in the pile or stack, thereby forming a stack height of the biomass. As shown in FIG. 2, the outermost layer 112a encloses the stack height of the biomass. The outermost layer 112a is primarily composed of the cover 112. Since the dry digester 110 does not necessarily require a particular stack height to be achieved for initiating anaerobic digestion of the biomass, the present invention can be utilized for any scale or quantity of biomass, i.e., industrial, or micro scale. Further, unlike the existing systems, the present invention does not require prior heating and/or prior mixing of the biomass before stacking in the dry digester 110. Furthermore, the biomass is also not required to be subjected to size reduction or is devoid of such a requirement prior to stacking in the dry digester 110. This reduces the complexity and capex as well as the operating cost of the system 100.

In an embodiment, the dry digester 110 is maintained at a temperature ranging between 20° C. to 40° C., and pH ranging between 6.0 to 8.0. Once suitable conditions for anaerobic digestion are achieved, the dry digester 110 generates leachate, digestate, and biogas.

In another embodiment, the ground sheet 111 and cover 112 are made of a fabric material. The fabric material comprises of butyl, polyethylene, and polyvinyl chloride. The ground sheet 111 and cover 112 are made of the same or different fabric material. Hence, most of the structure of the dry digester 110 is made from fabric material. This provides for mobility and reduced capex in the system 100 as compared to traditional anaerobic digesters made from concrete. Furthermore, this also enables the present invention to be easily dismantled and reassembled onsite, as and where needed.

The irrigation/port system 113 ensures that complete wetting of the biomass is attained. The irrigation/port system 113 includes a plurality of irrigation lines 113a disposed over the cover 112 on the outermost layer 112a of the dry digester 110. As shown in FIG. 2, the plurality of irrigation lines 113a are connected to the sump system 120 and the Hi-rate digester tank 170. The plurality of irrigation lines 113a are preferably tubes and/or pipes of suitable diameter that run across multiple entry points on the outermost layer 112a. The plurality of irrigation lines 113a are configured to carry out a dual role: introducing a liquid suspension into the dry digester 110 thereby wetting the lignocellulosic biomass, and recycling the leachate.

During wetting, the liquid suspension is uniformly distributed within the stack or pile of the biomass. This ensures complete breakdown of the biomass into biogas. The biological reactions taking place in the dry digester 110 include hydrolysis, acidogenesis, acetogenesis, and methanogenesis.

Hydrolysis denotes the initial degradation of the lignocellulosic biomass into simpler constituents. This biological reaction involves the enzymatic cleavage of complex organic polymers, such as cellulose and hemicellulose, into soluble sugars, oligomers, and other similar substances. The process of hydrolysis enhances the digestibility of the lignocellulosic biomass and is an essential precursor to subsequent stages of the anaerobic digestion.

In acidogenesis, the microbial fermentation of the hydrolyzed products generated during hydrolysis takes place. In this phase, the microbial community metabolizes the soluble sugars, oligosaccharides, and other intermediates produced during the hydrolysis step. This metabolic activity leads to the formation of organic acids, alcohols, volatile fatty acids (VFAs), and additional compounds. Acidogenesis contributes to the accumulation of intermediate metabolites, which serve as substrates for subsequent reaction stages.

Acetogenesis characterizes the biotransformation of the intermediate compounds produced during acidogenesis into acetate, hydrogen (H₂), and carbon dioxide (CO₂) by acetogenic microorganisms. This conversion is pivotal in the generation of acetate, a key precursor for methane production in the subsequent methanogenic stage. Acetogenesis plays a crucial role in maximizing the bioconversion of organic matter within the anaerobic digestion process.

Lastly, methanogenesis refers to the bioconversion of acetate, H2, and CO2, as well as other volatile organic compounds generated during the preceding phases, into methane (CH4) by methanogenic archaea. This phase results in the predominant production of biogas, primarily composed of CH4 and CO2.

In an embodiment, the liquid suspension comprises of a microbial community, nutrients, buffering agents, and water. Suitable microbial community in the present context comprise of hydrolytic bacteria, fermentative bacteria, obligate hydrogen producing bacteria, homoacetogenic bacteria, syntrophic acetate oxidising bacteria, acetoclastic methanogens, hydrogenotrophic methanogens, and denitrifying methanogens.

The dry digester 110 also includes a plurality of tubes (not shown in Figures) disposed on the ground sheet 111 for collecting water and/or leachate. The plurality of tubes is dispersed on the ground sheet 111 at multiple locations and connected to the bottom surface of the pile or stack of biomass. The plurality of tubes is connected with a sump return box 115 (not shown in Figures) for storing the collected water and/or leachate. The sump return box 115 is connected to the sump system 120. Water and/or leachate from the plurality of tubes flow to the sump return box 115 under the influence of gravity and is stored for a short duration before being transferred to the sump system 120, also under the influence of gravity.

Once the liquid suspension is introduced at the top surface of the pile or stack and/or the leachate is recycled, water and/or leachate received from the bottom surface of the pile or stack is collected on the ground sheet 111, thereby forming a liquid level 114 represented by dotted lines inside the dry digester 110, as shown in FIGS. 3 and 4. The liquid level 114 comprises water and/or leachate. However, the liquid level may also include other ingredients of the liquid suspension that may have trickled from the pile or stack of the biomass as well as pieces or chunks of biomass carried by the liquid suspension and/or leachate. The plurality of tubes is disposed within the liquid level 114 on the ground sheet 111, whereby water and/or leachate is collected and transported to the sump return box 115 and subsequently to the sump system 120, as mentioned above.

In another embodiment, the dry digester 110 also includes a ballast tube B disposed on the cover 112 and the ground sheet 111, as shown in FIG. 4. The ballast tube B is dispersed on periphery of the dry digester 110 on the ground sheet 111 and cover 112, thereby acting as a weight to the cover 112 and ground sheet 111. Therefore, when the ground sheet 111 and the cover 112 are pulled up, a tight seal is formed by the ballast tank B, which prevents leakage of biogas from the dry digester 110.

In an embodiment, the ballast tube B is a continuous tube covering the entire periphery of the dry digester 110 and disposed on the ground sheet 111 and the cover 112. In another embodiment, more than one ballast tube B may be disposed on the periphery of the dry digester 110 on the ground sheet 111 and the cover 112. Herein, the ballast tube B can be disconnected or connected to each of the ballast tube B to form a continuous ballast tube B on the periphery of the dry digester (110).

Since the dry digester 110 is primarily composed of fabric material, the structural stability of the dry digester 110 is ensured by a plurality of ground sheet poles 116 and a plurality of cover sheet poles 117. Each of the plurality of ground sheet poles 116 and plurality of cover sheet poles 117 are installed on the ground sheet 111 and cover 112 respectively. Said otherwise, each of the plurality of ground sheet poles 116 and the plurality of cover sheet poles 117 are fixed on the ground sheet 111 and the cover 112 in a horizontal orientation to ensure that the pile or stack of biomass remains stable and prevent leakage of leachate and/or water and/or biogas.

In an embodiment, the plurality of ground sheet poles 116 and the plurality of cover sheet poles 117 are laid adjacent to each other in a horizontal orientation. In another embodiment, the plurality of ground sheet poles 116 and the plurality of cover sheet poles 117 are laid over each other in the horizontal orientation. Herein, “orientation” is defined as the direction in which length of the pipe is placed. For instance, in horizontal orientation, both the ground sheet pole 116 and the cover sheet pole 117 are laid such that their lengths are parallel to the ground. The length of the pipe encompasses and connotes the linear dimension of the pipe along its longitudinal axis.

Referring to FIG. 2, a webbing 118 formed over the cover 112 in the dry digester 110 provides for the required tautness. The webbing 118 is formed by a plurality of ropes 119 which bind at least the plurality of ground sheet poles 116 with the plurality of cover sheet poles 117 in the manner shown in FIG. 4. The webbing 118 further extends to also cover multiple locations on the outermost layer 112a of the dry digester 110. In an embodiment, the plurality of ropes 119 are arranged at fixed separation between each of the rope 119 to form the webbing 118. In another embodiment, the plurality of ropes 119 are arranged at different separation between each of the rope 119 to form the webbing 118. The webbing 118 also ensures that the dry digester 110 is stable and that there is no leakage of leachate and/or water and/or biogas from the dry digester 110.

In an embodiment, as shown in FIG. 4, the cover 112 encloses the pile or stack of biomass from the top surface to the bottom surface on the ground sheet 111. The cover 112 and the ground sheet 111 being in contact with each other around the ballast tank B and at least partially wrapping the ballast tank B. The cover 112 and the ground sheet 111 connected to the plurality of ground sheet poles 116 and the plurality of cover sheet poles 117. The webbing 118 binding with the plurality of ground sheet poles 116 and the plurality of cover sheet poles 117 through the plurality of ropes 119. The arrangement described herein is repeated at multiple locations on the periphery of the dry digester 110 on the ground sheet 111 to ensure that the pile or stack of biomass (shown in FIG. 4) is tightly enclosed in the dry digester 110.

In an embodiment, the dry digester 110 is optionally subjected to seeding after wetting of biomass. Seeding is carried out in the presence of a seed material comprising manure, such as cow dung and buffalo dung. Other sources of manure or seed from other anaerobic digesters can also be used for seeding. By way of seeding, a deliberate and controlled introduction of specialized microbial consortia occurs, which is referred to as seed inoculum or biological starter culture. Seeding serves as a foundational step to expedite and optimize the establishment of requisite anaerobic microbial communities within dry digester 110.

Referring to FIG. 1, the sump system 120 monitors the leachate formed therein and circulated in the system 100. The sump system 120 comprises an external tank 122 connected to the dry digester 110. The external tank 122 stores the leachate. A sump tank 124 connected to the external tank 122 also stores the leachate. A pump 126 connected to the external tank 122 and the sump tank 124 circulates the leachate from the sump tank 124. The external tank 122 as a volume buffer for the sump tank 124.

In an embodiment, the leachate from the dry digester 110 is transported to the external tank 122 and/or the sump tank 124 under the influence of gravity. Said otherwise, the plurality of tubes in the liquid level 114 of the dry digester 110 collect water and/or leachate and further distribute the same to the external tank 122 as well as the sump tank 124 without using any external force, but due to gravitational force alone. The flow of water and/or leachate under the influence of gravity is attained by ensuring that each of the aforementioned components of the system 100 are arranged at different elevations. The present invention, therefore, minimizes the dependence on external factors and necessary means (such as pumps). This, in turn, reduces the overall capex and increases the mobility of the system 100.

The sump system 120 monitors the leachate for one or more parameters selected from pH, temperature, dissolved solids, micro elements, and macro elements. The parameters are monitored using suitable probes or devices that are connected to the sump system 120. Such probes or devices are known to the person skilled in the art. Herein, the micro elements include, but are not limited to, selenium, molybdenum, zinc, iron, and nickel. The macro elements include carbon and nitrogen. Other parameters that can be monitored in the sump system 120 include chemical oxygen demand (COD), volatile solids content and the volatile fatty acid profile, owing to the biological reactions in the dry digester 110. For this, a provision for sampling the leachate is provided in sump system 120. Therefore, the sump tank 120 ensures that the conditions preferable for anaerobic dry digestion are maintained in the leachate while it is being recycled back to the dry digester 110.

Referring to FIG. 1, a rotary valve 130 connects at least the pump 126 and the dry digester 110. The rotary valve 130 recycles the leachate from the sump tank 124 to the dry digester 110. As shown in FIG. 2, the plurality of irrigation lines 113a receive the recycled leachate and further introduce into the dry digester 110 through multiple entry points on the outermost layer 112a. Therefore, the microbial community is efficiently used, thereby maintaining the consistency of biology (and therefore biogas production) in different batches and further reduction in parasitic power requirement.

While the leachate is being recycled from the dry digester 110 to the sump system 120 and back to the dry digester 110, biogas generated in the system 100 is collected in a gas storage bladder 140 from various sources. As shown in FIG. 1, the primary source for biogas generation in the system remains the dry digester 110. However, during the movement of leachate from the dry digester 110 to the sump system 120, some amount of biogas gets entrapped and reaches the external tank 122 as well. Furthermore, a Hi-rate digester tank 170 is also connected to the rotary valve 130 for receiving the leachate. A bio-media is added in the Hi-rate digester tank 170 to improve biogas offtake from the leachate. The biogas from the Hi-rate digester tank 170 is also directed towards the gas storage bladder 140, along with the biogas from dry digester 110 and the external tank 122, as described herein.

In an embodiment, the rotary valve 140 recycles the leachate to each part of the digester through the irrigation lines in a sequenced, timed interval. Furthermore, the Hi-rate digester tank 170 may receive the leachate from the rotary valve in a sequenced, timed interval. The sequenced time intervals for recycling and sending the leachate to the dry digester 110 and the Hi-rate digester tank 170 may be same or different. The sequenced timed interval may constitute a cyclic pattern of leachate circulation. In an embodiment, the cyclic pattern may repeat itself in a user program sequence, such as by way of a computer program. The program sequence can be made dependent on geography as well as diurnal temperature variation. The cycle can be paused during low ambient temperatures (for example less than 15° C.) to prevent a temperature drop in the system 100.

In an embodiment, the bio-media comprises of a plastic material. In another embodiment, the bio-media comprises of extruded bioplastic. The person skilled in the art is aware of suitable plastic materials and/or bioplastics that can be used as suitable bio-media in the context of anaerobic digestion.

The gas storage bladder 140 is connected at least to a gas treatment unit 150 and a gas booster pump 160. The gas booster pump 160 assists the movement of biogas to the gas storage bladder and facilitates the measurement of biogas volume produced in the system 100. The gas booster pump 160 is connected at least to the dry digester 110 for pumping biogas through the gas treatment unit 150. Biogas generated in the dry digester 110 contains trace amounts of other impurities in gaseous form such as hydrogen sulfide, oxygen, ammonia, nitrogen, hydrogen, and volatile organic compounds. The gas treatment unit 150 receives biogas from the gas storage bladder 140 and removes a majority of these impurities.

In an embodiment, the gas treatment unit 150 includes one or more packed beds of suitable adsorbents for adsorbing at least hydrogen sulfide. For instance, the adsorbent can be activated carbon. The person skilled in the art is well aware of other suitable adsorbents that can be installed in the gas treatment unit 150 to adsorb the impurities.

In an embodiment, a condensate remover 180 is connected to the dry digester 110 and the gas booster pump 160. The condensate remover 180 receives biogas at least from the dry digester 110 and the Hi-rate digester tank 170 for removing moisture present in biogas, for example by chilling. In an embodiment, the condensate remover 180 receives biogas from the dry digester 110, the external tank 122, and the Hi-rate digester tank 170.

In an embodiment, the Hi-rate digester tank 170 is heated at a temperature ranging between 15° C. to 50° C., thereby resulting in an overflow of the leachate. This leachate is transported back to the sump tank 124, as shown in FIG. 1.

In another embodiment, the Hi-rate digester tank 170 comprises a control panel to monitor the biogas offtake and to further control the bio-media addition into the leachate. The control panel can be configured to measure, determine, and display at least the following parameters: timing and flow of irrigation, gas extraction, and onward consumption of gas. Suitable probes and/or devices for this purpose are known to the person skilled in the art which are typically connected to the control panel.

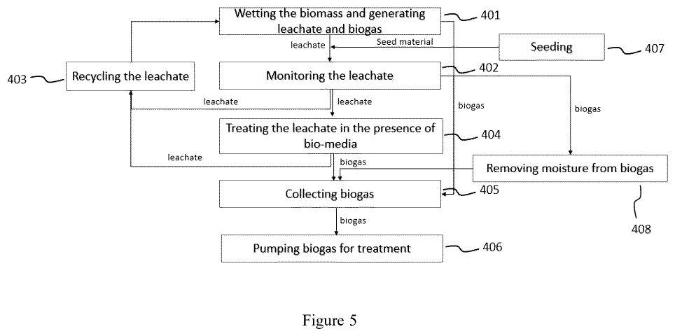

In another aspect, the present invention relates to a method for anaerobic dry digestion of lignocellulosic biomass. In this regard, reference is made to FIG. 5 which illustrates configuration of the system 100 and the method for anaerobic dry digestion of lignocellulosic biomass. Accordingly, the embodiments pertaining to the system 100, described hereinabove, are applicable here as well.

At step 401, wetting of the lignocellulosic biomass in the presence of liquid suspension takes place. The liquid suspension is introduced by the plurality of irrigation lines 113a disposed over the cover 112 of the dry digester. The plurality of irrigation lines 113a run across multiple entry points on the outermost layer 112a. Herein, the dry digester is maintained at a temperature ranging between 20° C. to 40° C., and pH ranging between 6.0 to 8.0.

Subsequently, the leachate generated during the digestion process is transferred to the sump system 120 under the influence of gravity. At step 402, monitoring of the leachate stored in the external tank 122 and the sump tank 124 is carried out. The leachate is constantly monitored for various parameters pH, temperature, dissolved solids, micro elements, and macro elements.

Optionally, at step 407, seeding of the biomass is carried out by addition of the seed material comprising manure. As the seed material already contains microorganisms from different sources, such as manure or seed from another anaerobic digester, there is an overall increase in the activity of the microbial community in the dry digester 110. This, in turn, accelerates the digestion process.

Subsequently, the leachate, at step 403, is recycled back to the dry digester 110 from the sump tank 124. The leachate recycling is carried out by the rotary valve 130 and is introduced back in the dry digester 110 by the plurality of irrigation lines 113a through multiple entry points, as depicted in FIG. 2.

Meanwhile, the leachate, at step 404, is subjected to treatment in the presence of bio-media at a temperature ranging between 15° C. to 50° C. to improve the biogas offtake. This is carried out by transporting the leachate to the Hi-rate digester tank 170. The temperature conditions in the Hi-rate digester tank 170 favor the offtake of biogas, however, resulting in an overflow of the leachate. The overflown leachate is transported back to the sump tank 124. Whereas the biogas is transported to the gas storage bladder 140.

At step 405, the gas storage bladder 140 receives biogas from the dry digester as well. Additionally, the leachate transported to the sump system 120 from the dry digester 110 also contains some amount of biogas entrapped therewith. This biogas is also transferred to the gas storage bladder 140.

The biogas generated in the dry digester 110 contains trace amounts of other impurities in gaseous form such as hydrogen sulfide, oxygen, ammonia, nitrogen, hydrogen, and volatile organic compounds. These impurities need to be removed before biogas is supplied for further consumption. For this, at step 406, the biogas is pumped by the gas booster pump 160 to the gas treatment unit 150. The gas treatment unit 150 receives biogas from the gas storage bladder 140 and removes a majority of these impurities. In an embodiment, the gas treatment unit 150 includes one or more packed beds of suitable adsorbents for adsorbing at least hydrogen sulfide. For instance, the adsorbent can be activated carbon for adsorbing hydrogen sulfide. The person skilled in the art is well aware of other suitable adsorbents that can be installed in the gas treatment unit 150 to adsorb the impurities.

Since biogas may also contain minor amounts of moisture primarily from the dry digester 110, it is pertinent to remove the moisture prior to subjecting biogas to the gas treatment unit 150. For this, at step 408, biogas received from the dry digester 110, external tank 122, and Hi-rate digester tank 170 is passed through the condensate remover 180. Biogas received from the gas treatment unit 150 is rich in methane and carbon dioxide.

In an embodiment, the biogas obtained from the dry digester 110 comprises methane and carbon dioxide in the volume ratio ranging between 50:50 to 60:40. In terms of weight ratio, biogas comprises methane and carbon dioxide in the ratio ranging between 1.0:2.5 to 1.0:2.8. Additionally, biogas may also contain other gases such as hydrogen sulfide (less than 0.02 vol. %) and oxygen (less than 0.5 vol. %). The biogas obtained from the gas treatment unit 150 is sent for further consumption, for e.g., in households and industry.

The digestate left in the dry digester 110 is collected on the ground sheet 111 and removed in a periodic or timely manner. This digestate is then used for various applications, such as but not limited to fertilizer, animal bedding, compost production, and the likes.

Advantageously, the present invention does not require any prior heating and/or prior mixing in the lignocellulosic biomass, before wetting. In contrast to the existing anaerobic digestion systems and methods, the present invention provides for a compact and easy to assemble and disassemble (has mobility) system, reduction in capital expenditure (i.e., cost effectiveness) and environmental impact, overcomes the inconsistent biology (and therefore biogas production) through different batches, reduces high parasitic power use by recycling of leachate, and provides for long retention times, thereby increasing the efficiency of biogas generation.

### REFERENCE NUMERALS

| | |
|---|---|
| 100 | System |
| 110 | Dry digester |
| 111 | Ground sheet |
| 112 | Cover |
| 112a | Outermost layer |
| 113 | irrigation/port system |
| 113a | irrigation lines |
| 114 | liquid level |
| B | ballast tube |
| 115 | Sump return box |
| 116 | ground sheet poles |
| 117 | cover sheet poles |
| 118 | webbing |
| 119 | ropes |
| 120 | Sump system |
| 122 | External tank |
| 124 | Sump tank |
| 126 | Pump |
| 130 | Rotary valve |
| 140 | gas storage bladder |
| 150 | Gas treatment unit |
| 160 | gas booster pump |
| 170 | hi-rate digester tank |
| 180 | Condensate remover |

### EXAMPLES

The following experimental examples are illustrative of the invention but not limitative of the scope thereof:

In an experimental setup, the dry digester 110 (having dimension of 19 m×19 m) was operated at ambient temperature which varied between 19° C. to 42° C. and ambient pressure (101.325 kPa). A mix of cow and buffalo manure was used as the seed material containing microorganisms. 10 tons of manure was added through 4 inch×2 inch tubes, along with water to fill the base of the dry digester 110 up to a height of 0.5 m, over a period of 1 week. The system 100 was designed to handle a total solids content of over 35%. During rice straw digestion, the total solids content in the system 100 was approximately 61%, which was higher than many conventional dry digesters. This minimises the use of water and its associated financial and environmental costs.

The cumulated biogas and methane yield collected from dry digestion of rice straw over 151 days is shown in FIG. 5. After 151 days, biogas and methane yield measured were 151 m³ and 75 m³ per ton of volatile solids (VS) of rice straw, respectively. The trend in biogas production is increasing, suggesting continued fermentation and methanogenesis of the substrate. The target methane yield production per day (of 200 m³) from rice straw could be achieved after this time. FIG. 5 also shows a lag phase before significant methane production was measured. This suggests that the use of the microbial community adapted for digesting rice straw may increase the methane production rate. When the digester was stopped, the Hi-rate digester tank 170 was able to retain and store the microorganisms adapted to digesting the substrate. Excess leachate was stored in an adjacent lagoon. Recycling the leachate to digest the next batch of substrates improves the consistency in biogas production in the batch system and enables a more rapid start-up phase.

The samples of the leachate were analysed on days 62, 98, and 124. The pH measured ranged between 7.9 and 8.2. An increase in the chemical oxygen demand and total trace element concentration (Table 1) over time suggests the solubilisation and break down of the rice straw. Additionally, the carbon-to-nitrogen ratio stabilised within the digester between days 62 and 98, without external intervention, from 105.1:1 to 24.1:1. The latter ratio was found to be more optimal for biogas production. This data highlights that the system 100 is capable of self-stabilising and the rice straw appears to be broken down to produce biogas without significant intervention.

TABLE 1
Total trace element content, chemical oxygen demand (COD) and
carbon-to-nitrogen (C:N) ratio of the leachate sampled at
three time points from the dry digester digesting rice straw.

| | Day 62 | Day 98 | Day 124 |
|---|---|---|---|
| Total trace | 5.67 | 6.78 | 13.74 |
| element content (g/L) | | | |
| COD (g/L) | 6.33 | 8.88 | 9.25 |
| C:N ratio | 105.10 | 24.05 | 22.70 |

The dry digester 110 was compared with a wet continuously stirred anaerobic digester known in the art. Table 2 below shows simulated data comparing wet anaerobic digesters and the present invention using the same input of 360 tonnes of rice straw.

TABLE 2
Comparison between the present invention and
conventional wet anaerobic digestion system

| | Dry System | Wet System |
|---|---|---|
| Target Biogas (m³ per ton) | 229.33 | 337.24 |
| Biomethane (m³ per ton) | 115.73 | 170.19 |
| Energy consumption (kWh per ton) | 51.56 | 170.59 |
| Heat consumption (kWh per ton) | — | 28.87 |

Capital Costs

| | | |
|---|---|---|
| Total cost | Total cost | Total cost |
| Costs (£ | Costs (£ | Costs (£ |
| per ton) | per ton) | per ton) |
| Costs of Biogas | Costs of Biogas | Costs of Biogas |
| produced (£ | produced (£ | produced (£ |
| per m³) | per m³) | per m³) |
| Costs of Biomethane | Costs of | Costs of |
| produced (£ | Biomethane | Biomethane |
| per m³) | produced (£ | produced (£ |
| | per m³) | per m³) |

Mass balance in kWh

| | | |
|---|---|---|
| 464,002 | 464,002 | 464,002 |
| 18,560 | 18,560 | 18,560 |
| 445,442 | 445,442 | 445,442 |
| 1,237 | 1,237 | 1,237 |
| 1 | 1 | 1 |

The conventional system produces 47% more biomethane per tonne of input than the present invention. The conventional system requires power for agitation and heat during its operation, consuming 37% more energy than the present invention. Additionally, the cost of the conventional system per m3 of biomethane produced is 2.1 times higher. The present invention can generate 73% of the energy produced by the wet system at 3.1 times lower total capital costs with approximately two weeks faster installation.

Thus, the present disclosure requires lower capital costs compared to conventional anaerobic digesters. Operational costs associated with power consumption are also reduced compared to conventional stirred tank reactors as the feedstock remains still (the leachate moves around). This also allows for longer retention times to break down the fibrous components of rice straw. The present invention allows its deployment at small scale and lower capital costs compared to conventional anaerobic digesters. For example, the capacity of conventional dry digesters ranges between 7,500-350,000 tons per annum, whereas the present invention can accommodate up to 100,000 tons per annum per system 100.

While the present invention has been described with respect to certain embodiments, it will be apparent to those skilled in the art that various changes and modification may be made without departing from the scope of the invention as defined in the following claims.

1. A system for anaerobic dry digestion of lignocellulosic biomass, comprising:
a dry digester comprising a ground sheet and a cover for the lignocellulosic biomass, said dry digester generating a leachate and biogas from anaerobic digestion,
an irrigation/port system for wetting of the lignocellulosic biomass under the cover, the irrigation/port system comprising a plurality of irrigation lines disposed over the cover and configured to:
introduce a liquid suspension into the dry digester thereby wetting the lignocellulosic biomass, the liquid suspension comprising of a microbial community, nutrients, buffering agents, and water, and
recycle the leachate,
a sump system for monitoring the leachate, the sump system comprising:
an external tank connected to the dry digester for storing the leachate,
a sump tank connected to the external tank for storing the leachate, and
a pump connected to the external tank and the sump tank, the pump circulating the leachate from the sump tank,
a rotary valve connected at least to the pump and the dry digester, the rotary valve recycling the leachate from the sump tank to the dry digester,
a gas storage bladder for collecting the biogas, the gas storage bladder connected at least to a gas treatment unit and a gas booster pump,
the gas booster pump connected at least to the dry digester and pumping biogas through the gas treatment unit, and
a hi-rate digester tank connected at least to the rotary valve for receiving the leachate therefrom, the hi-rate digester tank comprising a bio-media for improving biogas offtake from the leachate.

2. The system as claimed in claim 1, wherein the dry digester comprises:
a plurality of tubes disposed on the ground sheet for collecting water and/or leachate, the plurality of tubes connected with a sump return box for storing the collected water and/or leachate,
a plurality of ground sheet poles and a plurality of cover sheet poles installed on the ground sheet, said each ground sheet pole and each cover sheet pole providing structural stability to the dry digester,
a webbing formed over the cover for providing tautness to the dry digester, the webbing being formed by a plurality of ropes and binding at least the plurality of ground sheet poles with the plurality of cover sheet poles across the ground sheet, thereby preventing escape of biogas and leakage of the leachate.

3. The system as claimed in claim 1, wherein the lignocellulosic biomass is stacked on the ground sheet to form a pile of the lignocellulosic biomass having a top surface and a bottom surface, the cover enclosing the pile from the top surface to the bottom surface on the ground sheet.

4. The system as claimed in claim 1, wherein a ballast tube B is disposed on the cover and the ground sheet, the ballast tube B being dispersed on a periphery of the dry digester on the ground sheet and the cover, thereby acting as a weight to the cover and the ground sheet and sealing the dry digester to prevent leakage of biogas.

5. The system as claimed in claim 1, wherein the dry digester is maintained at a temperature ranging between 20° C. to 40° C., and pH ranging between 6.0 to 8.0.

6. The system as claimed in claim 1, wherein the wetting allows the microbial community to uniformly distribute within the lignocellulosic biomass, thereby ensuring complete breakdown of the lignocellulosic biomass into biogas via hydrolysis, acidogenesis, acetogenesis, and methanogenesis.

7. The system as claimed in claim 1, wherein the leachate from the dry digester is transported to the external tank and/or the sump tank under the influence of gravity.

8. The system as claimed in claim 1, wherein the hi-rate digester tank is heated at a temperature ranging between 15□C to 50□C, thereby resulting in an overflow of the leachate, said leachate being transported back to the sump tank.

9. The system as claimed in claim 1, comprising a condensate remover connected to the dry digester and the gas booster pump, the condensate remover receiving biogas at least from the dry digester and the hi-rate digester tank for removing moisture present in the biogas.

10. The system as claimed in claim 1, wherein the ground sheet and the cover, independent of each other, comprises of butyl, polyethylene, and polyvinyl chloride.

11. The system as claimed in claim 1, wherein the leachate is monitored for one or more of parameters selected from pH, temperature, dissolved solids, micro elements, and macro elements.

12. A method for anaerobic dry digestion of lignocellulosic biomass, said method comprising:
(a) wetting, in a dry digester, the lignocellulosic biomass in the presence of a liquid suspension introduced by a plurality of irrigation lines disposed over a cover of the dry digester, thereby generating a leachate and biogas,
wherein the dry digester is maintained at a temperature ranging between 20° C. to 40° C., and pH ranging between 6.0 to 8.0,
(b) monitoring, in a sump system comprising an external tank and a sump tank, the leachate stored in the external tank and the sump tank, the leachate being transported to the external tank and the sump tank under the influence of gravity,
(c) recycling, by a rotary valve, the leachate from the sump tank to the dry digester, the leachate being introduced in the dry digester by the plurality of irrigation lines,
(d) treating, in a hi-rate digester tank, the leachate received from the rotary valve in the presence of a bio-media and at a temperature ranging between 15° C. to 50° C., thereby improving the biogas offtake from the leachate,
(e) collecting, in a gas storage bladder, biogas received from the dry digester, and
(f) pumping, by a gas booster pump, biogas to a gas treatment unit, thereby removing at least hydrogen sulphide from the biogas.

13. The method as claimed in claim 12, wherein in the dry digester optionally seeding is carried out after the wetting of the lignocellulosic biomass, the seeding being carried out in the presence of a seed material comprising manure.

14. The method as claimed in claim 12, wherein the liquid suspension comprises of a microbial community, nutrients, buffering agents, and water.

15. The method as claimed in claim 12, wherein prior to collecting biogas in the gas storage bladder, biogas received at least from the dry digester and the Hi-rate digester tank is subjected to moisture removal, in a condensate remover.

16. The method as claimed in claim 12, wherein lignocellulosic biomass is selected from rice straw, wheat straw, bagasse, corn stover, Miscanthus, Napier grass, and palm fronds.

17. The system as claimed in claim 12, wherein the microbial community comprises of hydrolytic bacteria, fermentative bacteria, obligate hydrogen producing bacteria, homoacetogenic bacteria, syntrophic acetate oxidising bacteria, acetoclastic methanogens, hydrogenotrophic methanogens, and denitrifying methanogens.

18. The system as claimed in claim 12, wherein the bio-media comprises extruded bioplastic.
